(19) United States
(12) Patent Application Publication
(10) Pub. No.: US 2024/0081659 A1
(43) Pub. Date: Mar. 14, 2024

(54) BIOLOGICAL INFORMATION MEASUREMENT SYSTEM, BIOLOGICAL INFORMATION MEASUREMENT RECORDING MEDIUM, AND BIOLOGICAL INFORMATION MEASUREMENT DEVICE

(71) Applicant: OMRON HEALTHCARE CO., LTD., Kyoto (JP)

(72) Inventors: Kentaro MORI, Kyoto (JP); Takeshi KUBO, Kyoto (JP); Yasuo ASANO, Kyoto (JP); Yoshihide TOKKO, Kyoto (JP)

(21) Appl. No.: 18/511,499

(22) Filed: Nov. 16, 2023

Related U.S. Application Data
(63) Continuation of application No. PCT/JP2022/022294, filed on Jun. 1, 2022.

(30) Foreign Application Priority Data
Jun. 25, 2021 (JP) 2021-106011

(51) Int. Cl. A61B 5/0205; A61B 5/00

(52) U.S. Cl. CPC A61B 5/0205; A61B 5/7221; A61B 5/7225; A61B 5/7445; A61B 5/021; A61B 2560/045

(57) ABSTRACT

A biological information system/device/program/recording medium, which may include: a biological information measurement device; and a terminal. The biological information measurement device includes: a measurement unit that can measure first information related to a living body and second information related to the living body; a first processing unit that can execute first processing related to display or diagnosis on the first information; and a communication unit that can communicate the second information with the terminal. The terminal includes: a reception unit that can receive the second information from the biological information measurement device; a second processing unit that can execute second processing related to display or diagnosis on the second information; and a transmission unit that can transmit the second information on which the second processing is executed to the biological information measurement device.

### CROSS-REFERENCE TO RELATED APPLICATIONS

This application is the U.S. national stage application filed pursuant to 35 U.S.C. 365(c) and 120 as a continuation of International Patent Application No. PCT/JP2022/022294, filed Jun. 1, 2022, which application claims priority to Japanese Patent Application No. 2021-106011, filed Jun. 25, 2021, which applications are incorporated herein by reference in their entireties.

### TECHNICAL FIELD

The present invention relates to a biological information measurement system, a biological information measurement program (recording medium), and a biological information measurement device.

### BACKGROUND ART

As a device that measures a plurality of pieces of biological information and processes the biological information to determine a state of a living body, for example, a biological information measurement device is disclosed (for example, Patent Document 1) that measures a blood pressure value and a waveform of a cardiac action potential and determines a possibility of changes in physical condition of a measurement subject from the measurement result.

### CITATION LIST

### Patent Literature

Patent Document 1: JP 2017-176340 A

### SUMMARY OF INVENTION

### Technical Problem

For example, as the processing of the waveform of the cardiac action potential, filtering processing for removing noise included in the waveform, determination processing for determining whether a cardiac function is normal by a predetermined algorithm, and the like can be considered. However, relatively large computational costs are required to execute such processing. Therefore, in order to enhance an arithmetic function, the biological information measurement device needs to be provided with a high-performance processor. As a result, it is assumed that the size of the biological information measurement device increases or power consumption increases. Therefore, it may be difficult to carry the biological information measurement device and convenience may decrease.

The present invention is made in view of such circumstances, and an object thereof is to provide techniques that suppress a decrease in convenience of a biological information measurement device and enable a plurality of pieces of biological information to be analyzed.

### Solution to Problem

The present invention adopts the following configurations in order to achieve the above-mentioned object.

In other words, a biological information measurement system according to an aspect of the present invention includes a biological information measurement device and a terminal that is portable. The biological information measurement device includes: a measurement unit configured to measure at least first information related to a living body and second information related to the living body; a first processing unit configured to execute first processing related to display and/or diagnosis on the first information measured by the measurement unit; and a communication unit configured to communicate the second information with the terminal. The terminal includes: a reception unit configured to receive the second information from the biological information measurement device; a second processing unit configured to execute second processing related to display and/or diagnosis on the second information received by the reception unit; and a transmission unit configured to transmit the second information after the second processing is executed by the second processing unit to the biological information measurement device. The biological information measurement device further includes a first display unit configured to display the first information after the first processing is executed by the first processing unit and the second information transmitted to the biological information measurement device by the transmission unit after the second processing is executed by the second processing unit.

Here, the first processing related to display includes arithmetic processing based on the first information measured by the measurement unit. Further, the second processing related to display includes filter processing on the second information measured by the measurement unit.

Furthermore, the first processing related to diagnosis includes determination processing that can diagnose the health condition of a measurement subject based on the first information measured by the measurement unit. In addition, the second processing related to diagnosis includes determination processing that can diagnose the health condition of the measurement subject based on the second information measured by the measurement unit.

With such a configuration, a plurality of pieces of biological information can be measured and analyzed. In addition, according to such a configuration, computational costs of the biological information measurement device are reduced compared with a case where the second processing is executed in the biological information measurement device. Therefore, an increase in size of a processor mounted in the biological information measurement device and an increase in power consumption can be suppressed. As a result, the biological information measurement device can be easily carried, and convenience is improved.

In the biological information measurement system according to the aspect described above, the terminal may further include a second display unit configured to display the second information on which the second processing is executed by the second processing unit.

With such a configuration, the measurement subject can check the second information from the terminal even when the measurement subject does not carry the biological information measurement device.

Further, a biological information measurement program according to an aspect of the present invention may cause a computer to execute: a measuring step of measuring at least first information related to a living body and second information related to the living body in a biological information measurement device; a first processing step of executing first processing related to display and/or diagnosis on the first information measured in the measuring step in the biological information measurement device; a first transmitting step of transmitting the second information to a terminal that is portable in the biological information measurement device; a first receiving step of receiving, in the terminal, the second information transmitted from the biological information measurement device in the first transmitting step; a second processing step of executing, in the terminal, second processing related to display and/or diagnosis on the second information received in the first receiving step; a second transmitting step of transmitting, in the terminal, the second information on which the second processing is executed in the second processing step to the biological information measurement device; a second receiving step of receiving, in the biological information measurement device, the second information on which the second processing is executed, the second information transmitted from the terminal in the second transmitting step; and a displaying step of displaying, in the biological information measurement device, the first information after the executing the first processing and the second information after the executing the second processing received in the second receiving step.

Furthermore, a biological information measurement device according to an aspect of the present invention may include: a measurement unit configured to measure at least first information related to a living body and second information related to the living body; a first processing unit configured to execute first processing related to display and/or diagnosis on the first information measured by the measurement unit; a communication unit configured to communicate the second information with an external device including a second processing unit configured to execute second processing related to display and/or diagnosis on the second information; and a display unit configured to display the first information on which the first processing is executed by the first processing unit and the second information after execution of the second processing by the external device.

In the biological information measurement device according to the aspect described above, the first information may include blood pressure information, and the second information may include cardiac action potential information.

Here, the second processing includes filtering processing for removing noise included in the waveform of the cardiac action potential and determination processing for determining whether a cardiac function is normal.

With such a configuration, the blood pressure and the cardiac action potential can be measured and analyzed. In addition, with such a configuration, computational costs of the biological information measurement device are reduced compared with a case where the second processing is executed in the biological information measurement device. Therefore, the size of the processor mounted in the biological information measurement device can be reduced. As a result, an increase in size of the biological information measurement device is suppressed.

In the biological information measurement device according to the aspect described above, the second processing may include filtering processing on a waveform of the cardiac action potential and determination processing related to a cardiac function.

With such a configuration, the blood pressure and the cardiac action potential can be measured and analyzed. In addition, with such a configuration, computational costs of the biological information measurement device are reduced compared with a case where the second processing is executed in the biological information measurement device. Therefore, the size of the processor mounted in the biological information measurement device can be reduced. As a result, an increase in size of the biological information measurement device is suppressed.

In the biological information measurement device according to the aspect described above, the second information may include at least one piece of information about blood pressure, a pulse wave, percutaneous arterial blood oxygen saturation, an action potential generated from a muscle fiber, acceleration of the biological information measurement device in a predetermined direction, transmission or reflection intensity of light emitted from the biological information measurement device, ambient temperature of the biological information measurement device, or body temperature of a measurement subject on which the biological information measurement device is attached.

With such a configuration, the biological information and the second information affecting the biological information can be measured and analyzed. In addition, with such a configuration, computational costs of the biological information measurement device are reduced compared with a case where the second processing is executed in the biological information measurement device. Therefore, the size of the processor mounted in the biological information measurement device can be reduced. Therefore, an increase in size of the biological information measurement device that measures information related to a plurality of pieces of biological information is suppressed.

In the biological information measurement device according to the aspect described above, the display unit may synchronize timing of displaying the first information on which the first processing is executed by the first processing unit and timing of displaying the second information on which the second processing is executed by the second processing unit.

With such a configuration, two different pieces of first information and second information can be checked at the same time. As a result, convenience is improved.

### Advantageous Effects of Invention

According to the present invention, techniques that suppress a decrease in convenience of a biological information measurement device while enabling a plurality of pieces of biological information to be analyzed can be provided.

### BRIEF DESCRIPTION OF DRAWINGS

FIG. 1 illustrates an overview of the configuration of a biological information measurement system according to an embodiment.

FIG. 2 illustrates an overview of the hardware configuration of a biological information measurement device.

FIG. 3 illustrates an overview of the functional configuration of the biological information measurement device and a smartphone.

FIG. 4 illustrates an overview of a blood pressure measurement flow in the biological information measurement device.

FIG. 5 illustrates an overview of a measurement flow of a cardiac action potential in the biological information measurement device.

FIG. 6 illustrates an overview of a display flow of a cardiac action potential in the smartphone.

FIG. 7 illustrates an example of a biological information measurement flow executed by the entire biological information measurement system.

FIG. 8 illustrates an overview of a display of the biological information measurement device on which information during measurement is displayed.

FIG. 9 illustrates an overview of the display of the biological information measurement device on which a systolic blood pressure value, a diastolic blood pressure value, a pulse rate, and a determination result of a cardiac function indicator are displayed.

FIG. 10 illustrates an overview of a display of the smartphone on which a waveform of the cardiac action potential and a determination result are displayed.

### DESCRIPTION OF EMBODIMENTS

### § 1 Embodiment

### System Summary

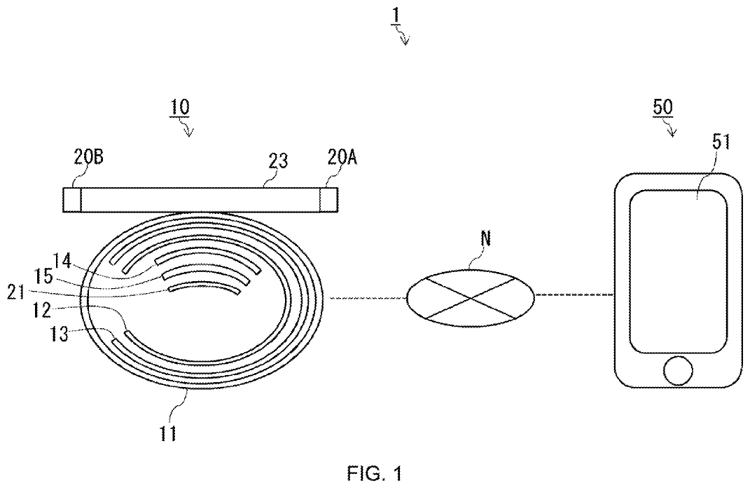

FIG. 1 illustrates an overview of the configuration of a biological information measurement system 1 according to the present embodiment. As illustrated in FIG. 1, the biological information measurement system 1 includes a biological information measurement device 10 and a smartphone 50. Note that the smartphone 50 may be replaced with a portable terminal such as a tablet terminal.

Also, in the biological information measurement system 1, the biological information measurement device 10 and the smartphone 50 are connected to each other via a network (N). The network (N) includes a wireless communication network, for example, a Wi-Fi (trade name) or a telephone communication network such as a mobile phone. Further, the network (N) includes wired communication connected via a local area network (LAN) cable. Furthermore, the network (N) may include, for example, a wide area network (WAN) that is a worldwide public communication network such as the Internet. In addition, as a replacement for the network (N), the biological information measurement device 10 and the smartphone 50 may be connected to each other via a universal serial bus (USB) cable. Moreover, as the replacement for the network (N), wireless communication may be performed between the biological information measurement device 10 and the smartphone 50 according to the Bluetooth (trade name) standard.

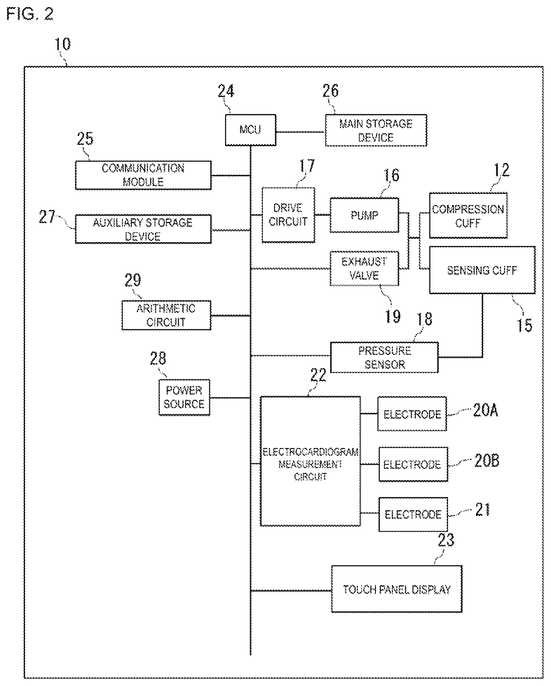

FIG. 2 illustrates an overview of the hardware configuration of the biological information measurement device 10 constituting the biological information measurement system 1 according to the present embodiment. Hereinafter, the hardware configuration of each of the biological information measurement device 10 and the smartphone 50 will be described in detail with reference to FIG. 1 or FIG. 2.

Hardware Configuration of Biological Information Measurement Device 10

As illustrated in FIG. 1 and FIG. 2, the biological information measurement device 10 includes a belt 11 that can be wound around the arm of a measurement subject. Further, the biological information measurement device 10 includes a compression cuff 12 that is a bag-shaped member disposed inside the belt 11 along the longitudinal direction of the belt 11. Furthermore, the biological information measurement device 10 includes a pump 16 that is connected to the compression cuff 12 and inflates the compression cuff 12 by sending air into the compression cuff 12, and an exhaust valve 19 that can discharge air inside the pump 16. The compression cuff 12 is inflated and thus can apply external pressure to the blood vessel of the measurement subject.

Further, the biological information measurement device 10 includes a sensing cuff 15 further on the inner side than the compression cuff 12. The sensing cuff 15 is a bag-shaped member, and air is fed into the sensing cuff 15 by the pump 16 and is discharged from the exhaust valve 19 in the same way as the compression cuff 12. Also, the compression cuff 12 is inflated and thus the sensing cuff 15 is pressed against the arm of the measurement subject. Furthermore, the biological information measurement device 10 includes a pressure sensor 18. The pressure sensor 18 is disposed to be able to measure internal pressure of the sensing cuff 15.

Further, the biological information measurement device 10 includes a curler 13 between the belt 11 and the compression cuff 12. The curler 13 functions as a base for holding the compression cuff 12. Furthermore, the biological information measurement device 10 includes a back plate 14. The back plate 14 is disposed between the compression cuff 12 and the sensing cuff 15. The back plate 14 suppresses excessive bending of the sensing cuff 15.

Further, the biological information measurement device 10 includes an electrode 21. The electrode 21 is disposed further on the inner side than the sensing cuff 15. When the belt 11 is wound around the arm of the measurement subject, the electrode 21 is brought into contact with the arm of the measurement subject.

Furthermore, the biological information measurement device 10 includes a touch panel display 23 on the outer surface of the belt 11, and an electrode 20A and an electrode 20B that are disposed on the outer periphery of the touch panel display 23.

In addition, although not illustrated in FIG. 1, the biological information measurement device 10 is a computer including, in addition to the aforementioned components, a micro controller unit (MCU) 24, a communication module 25 that can communicate according to predetermined communication standards, a main storage device 26 such as a random access memory (RAM) or a read only memory (ROM), an auxiliary storage device 27 such as a hard disk drive, a drive circuit 17 for driving the pump 16, an arithmetic circuit 29, an electrocardiogram measurement circuit 22 connected to the electrode 20A, the electrode 20B and the electrode 21 and configured to measure a potential difference between the electrode 20A with a potential of the electrode 20B as a reference potential and the electrode 21, and a power supply 28 configured to supply power necessary for operation such as measurement of a cardiac action potential. An operating system (OS), various programs, various tables, and the like are stored in the auxiliary storage device. In addition, programs stored in the auxiliary storage device 27 are loaded into a workspace of the main storage device 26 and executed, and each component and the like are controlled by execution of the programs. Thus, each function that meets a predetermined purpose as described below can be accomplished. Hereinafter, the main storage device 26 and the auxiliary storage device 27 will be simply referred to as a storage device.

Hardware Configuration of Smartphone 50

The smartphone 50 is a portable terminal including a processor such as a central processing unit (CPU), a storage device such as a RAM or a ROM, a camera, a touch panel display 51, a communication module, and an arithmetic circuit. An operating system (OS), various programs, various tables, and the like are stored in the storage device, and programs stored therein are loaded into a workspace of a main storage device and executed, and each component and the like are controlled by execution of the programs. Thus, each function that meets a predetermined purpose as described below can be accomplished. Note that the processor included in the smartphone 50 has a higher arithmetic function such as a calculation size or a calculation speed than an arithmetic function of an MCU 24 of the biological information measurement device 10.

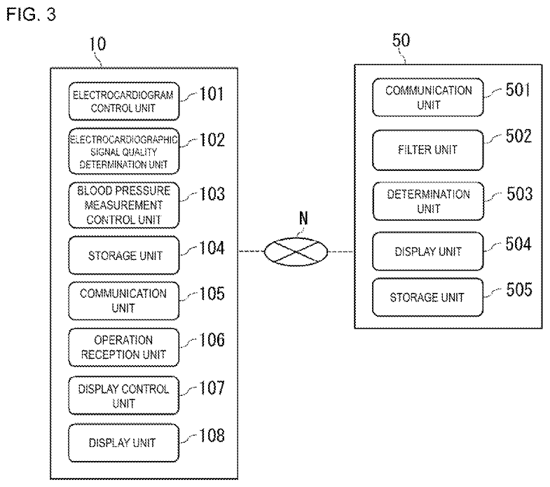

FIG. 3 illustrates an overview of the functional configuration of the biological information measurement device 10 and the smartphone 50.

Functional Configuration of Biological Information Measurement Device 10

As illustrated in FIG. 3, the biological information measurement device 10 includes an electrocardiogram control unit 101. The electrocardiogram control unit 101 receives input operation information of the touch panel display 23 from an operation reception unit 106 (described below). The input operation information is, for example, an electrical signal generated when the finger touches the touch panel display 23. Further, the electrocardiogram control unit 101 controls components such as a switch disposed in the electrocardiogram measurement circuit 22. Furthermore, the electrocardiogram control unit 101 instructs a storage unit 104 (described below) to store cardiac action potential information in the storage device. In addition, the electrocardiogram control unit 101 instructs a display control unit 107 (described below) to display a measurement status on a cardiac action potential on the touch panel display 23. The electrocardiogram control unit 101 is an example of a “measurement unit” of the present disclosure. The cardiac action potential information is an example of “second information” of the present disclosure.

Furthermore, the biological information measurement device 10 includes an electrocardiographic signal quality determination unit 102. The electrocardiographic signal quality determination unit 102 acquires, from the electrocardiogram measurement circuit 22, potential difference information between the electrode 20A with a potential of the electrode 20B as a reference potential and the electrode 21. Further, the electrocardiographic signal quality determination unit 102 determines in accordance with a predetermined algorithm whether the acquired potential difference information can be analyzed. The predetermined algorithm is, for example, an algorithm that enables analysis when the waveform of the potential difference has a predetermined frequency spectrum. Furthermore, when it is determined that the potential difference information can be analyzed, the electrocardiographic signal quality determination unit 102 inputs the potential difference information into a communication unit 105 (described below).

Further, the biological information measurement device 10 includes a blood pressure measurement control unit 103. The blood pressure measurement control unit 103 receives input operation information of the touch panel display 23 from the operation reception unit 106 (described below). Furthermore, the blood pressure measurement control unit 103 controls the driving of the pump 16 and the valve opening degree of the exhaust valve 19. In addition, the blood pressure measurement control unit 103 acquires internal pressure of the sensing cuff 15 via the pressure sensor 18. Moreover, the blood pressure measurement control unit 103 calculates a systolic blood pressure value, a diastolic blood pressure value, and a pulse rate based on the internal pressure of the sensing cuff 15 by using the arithmetic circuit 29 (an example of “first processing related to display” of the present disclosure). A method of calculating the systolic blood pressure value, the diastolic blood pressure value, and the pulse rate is a known algorithm or a uniquely developed algorithm. Further, the blood pressure measurement control unit 103 instructs the storage unit 104 (described below) to store the internal pressure of the sensing cuff 15, the systolic blood pressure value, the diastolic blood pressure value, and the pulse rate in the storage device. Furthermore, the blood pressure measurement control unit 103 instructs the display control unit 107 (described below) to display the internal pressure of the sensing cuff 15, the systolic blood pressure value, the diastolic blood pressure value, and the pulse rate on the touch panel display 23. Note that the blood pressure measurement control unit 103 is an example of the “measurement unit” and a “first processing unit” of the present disclosure. Additionally, the internal pressure of the sensing cuff 15 is an example of “first information” of the present disclosure.

Alternatively, the blood pressure measurement control unit 103 may determine whether the measurement subject is healthy by comparing the calculated systolic blood pressure value, diastolic blood pressure value, or pulse rate with a predetermined reference value (an example of “first processing related to diagnosis” of the present disclosure). Then, by executing the processing described below, the determined information about whether the measurement subject is healthy may be displayed on the touch panel display 23. Note that the diagnosis refers to diagnosis of a health state of the measurement subject. In addition to the above processing, processing that can determine whether the measurement subject is healthy may be executed.

Further, the biological information measurement device 10 includes the storage unit 104. The storage unit 104 includes the storage device. The storage unit 104 receives an instruction from another functional unit such as the electrocardiogram control unit 101 to store predetermined information in the storage device. The predetermined information includes, for example, blood pressure measurement information such as internal pressure of the sensing cuff 15, a systolic blood pressure value, a diastolic blood pressure value, and a pulse rate. Additionally, the predetermined information includes measurement information of the heart such as cardiac action potential information and a determination result of a cardiac function indicator. Note that the cardiac function indicator is, for example, cardiac rhythm or a heart rate. Further, the determination of the cardiac rhythm is, for example, to determine whether the frequency spectrum detected from the waveform of the cardiac action potential is the spectrum of a healthy person. Furthermore, the determination of the heart rate is, for example, to determine whether the heart rate measured in a predetermined period is within a predetermined range or higher than a predetermined value.

Further, the biological information measurement device 10 includes the communication unit 105. The communication unit 105 includes a communication module. The communication unit 105 communicates information with an external device according to predetermined communication standards. More specifically, the communication unit 105 transmits a connection request signal to the external device. When an acceptance signal of the connection request is received from the external device, predetermined transmission information is transmitted to the external device. The predetermined transmission information includes, for example, cardiac action potential difference information and information indicating that the measurement of the cardiac action potential is finished. Additionally, the communication unit 105 receives predetermined reception information from the external device. The predetermined reception information includes, for example, information related to the determination result of the cardiac function indicator. Note that the communication unit 105 is an example of a “communication unit” of the present disclosure.

Furthermore, the biological information measurement device 10 includes the operation reception unit 106. The operation reception unit 106 receives an operation input of the touch panel display 23. Then, the operation reception unit 106 notifies other functional units (for example, the blood pressure measurement control unit 103 and the electrocardiogram control unit 101) of the operation input information.

In addition, the biological information measurement device 10 includes the display control unit 107. The display control unit 107 receives from another control unit (for example, the blood pressure measurement control unit 103 or the like) an instruction to display predetermined display information on the touch panel display 23. Then, the display control unit 107 generates predetermined display information or reads display information from the storage device. Further, the display control unit 107 generates display control signals for controlling the timing of displaying the predetermined display information and the display area of the touch panel display 23. The display control signal that controls the timing of displaying the predetermined display information includes a signal for synchronizing the timing of displaying a plurality of pieces of display information or a signal for alternating the timing of displaying a plurality of pieces of display information. Alternatively, the display control unit 107 generates a display control signal for lighting up a predetermined area of the touch panel display 23. In addition, the display control unit 107 transmits the predetermined display information and the control signal to a display unit 108 (described below).

Further, the biological information measurement device 10 includes the display unit 108. The display unit 108 includes the touch panel display 23. The display unit 108 receives the predetermined display information and the display control signal from the display control unit 107. Then, the display unit 108 displays the predetermined display information on the touch panel display 23 in accordance with the display control signal. Note that the display control unit 107 and the display unit 108 are examples of a “display unit” and a “first display unit” of the present disclosure.

Functional Configuration of Smartphone 50

Next, each functional unit of the smartphone 50 will be described. As illustrated in FIG. 3, the smartphone 50 includes a communication unit 501. The communication unit 501 includes a communication module. The communication unit 501 communicates information with an external device according to predetermined communication standards. More specifically, the communication unit 501 receives a connection request signal from the external device. Then, the communication unit 501 transmits a signal for accepting the connection request to the external device. Then, the communication unit 501 transmits and receives predetermined information to and from the external device. Note that the communication unit 501 is an example of a “reception unit” and a “transmission unit” of the present disclosure.

Further, the smartphone 50 includes a filter unit 502. The filter unit 502 executes filtering processing on the waveform information to remove a noise component in the waveform of the cardiac action potential (an example of “second processing related to display” of the present disclosure). Note that the filter unit 502 is an example of a “second processing unit” of the present disclosure.

Furthermore, the smartphone 50 includes a determination unit 503. The determination unit 503 acquires a frequency spectrum from the waveform information of the cardiac action potential by analysis with a predetermined algorithm. Then, heart rate information is acquired based on the frequency spectrum. In addition, whether the cardiac rhythm is abnormal is determined based on the frequency spectrum (an example of “second processing related to diagnosis” of the present disclosure). Note that the predetermined algorithm includes, for example, fast Fourier transform (FFT) analysis. Note that the determination unit 503 is an example of the “second processing unit” of the present disclosure.

In addition, the smartphone 50 includes a display unit 504. The display unit 504 includes the touch panel display 51. The display unit 504 displays display information on the touch panel display 51. Additionally, the display unit 504 controls an area where the display information is displayed. Note that the display information includes the waveform of the cardiac action potential and the determination result of the cardiac function indicator. Note that the display unit 504 is an example of a “second display unit” of the present disclosure.

In addition, the smartphone 50 includes a storage unit 505. The storage unit 505 includes a storage device. The storage unit 505 stores predetermined information in the storage device.

Operation Flow of Each Device

Next, the overview of an operation flow in each of the biological information measurement device 10 and the smartphone 50 will be described.

Blood Pressure Measurement Flow in Biological Information Measurement Device 10

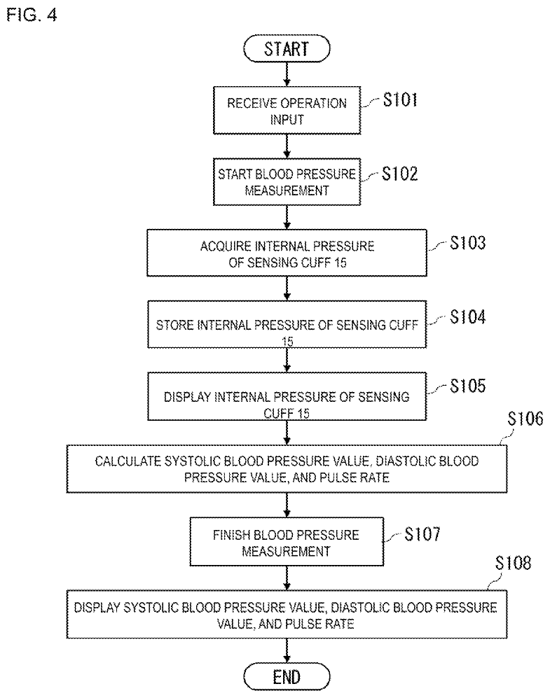

FIG. 4 illustrates an overview of a blood pressure measurement flow in the biological information measurement device 10.

S101

In step S101, the operation reception unit 106 receives an operation input of the touch panel display 23. Then, the operation reception unit 106 notifies the blood pressure measurement control unit 103 that the operation input has been performed.

S102

In step S102, the measurement of blood pressure is started. More specifically, the blood pressure measurement control unit 103 notified of the operation input in step S101 generates a control signal for controlling the valve opening degree of the exhaust valve 19. Then, the blood pressure measurement control unit 103 generates a drive signal for the compression cuff 12 and the sensing cuff 15. Thereafter, the blood pressure measurement control unit 103 transmits the generated control signal and drive signal to the exhaust valve 19 and the pump 16.

S103

In step S103, when the pump 16 receives the drive signal generated in step S102, the pump 16 is driven in accordance with the drive signal. Further, when the exhaust valve 19 receives the control signal generated in step S102, the valve opening degree of the exhaust valve 19 is controlled to a predetermined opening degree in accordance with the control signal. Furthermore, the blood pressure measurement control unit 103 acquires internal pressure of the sensing cuff 15 via the pressure sensor 18.

S104

In step S104, the blood pressure measurement control unit 103 instructs the storage unit 104 to store, in the storage device, the internal pressure of the sensing cuff 15 sequentially acquired via the pressure sensor 18 in step S103. Upon receiving the instruction, the storage unit 104 stores the internal pressure of the sensing cuff 15 in the storage device.

S105

In step S105, the blood pressure measurement control unit 103 instructs the display control unit 107 to display, on the touch panel display 23, the internal pressure of the sensing cuff 15 sequentially acquired via the pressure sensor 18 in step S103. The display control unit 107 receives internal pressure information of the sensing cuff 15 from the blood pressure measurement control unit 103 and instructs the display unit 108 to display the internal pressure information in a predetermined area of the touch panel display 23. Then, upon receiving the instruction from the display control unit 107, the display unit 108 displays the internal pressure information of the sensing cuff 15 on the touch panel display 23.

S106

In step S106, the blood pressure measurement control unit 103 calculates a systolic blood pressure value, a diastolic blood pressure value, and a pulse rate in a predetermined period by using the internal pressure of the sensing cuff 15 sequentially acquired via the pressure sensor 18 in step S103. A known algorithm or a uniquely developed algorithm is used to calculate the systolic blood pressure value, the diastolic blood pressure value, and the pulse rate in the predetermined period from the internal pressure of the sensing cuff 15. In addition, the blood pressure measurement control unit 103 instructs the storage unit 104 to store the calculated systolic blood pressure value, diastolic blood pressure value, and pulse rate in the storage device. Upon receiving the instruction, the storage unit 104 stores the systolic blood pressure value, the diastolic blood pressure value, and the pulse rate in the storage device.

S107

In step S107, when the blood pressure measurement control unit 103 calculates the systolic blood pressure value and the diastolic blood pressure value, the blood pressure measurement control unit 103 determines that the blood pressure measurement is finished. Then, the blood pressure measurement control unit 103 instructs the display control unit 107 to display, on the touch panel display 23, the systolic blood pressure value, the diastolic blood pressure value, and the pulse rate stored in the storage device.

S108

In step S108, the display control unit 107 that has received the instruction from the blood pressure measurement control unit 103 in step S107 instructs the display unit 108 to read out the systolic blood pressure value, the diastolic blood pressure value, and the pulse rate from the storage device and to display the systolic blood pressure value, the diastolic blood pressure value, and the pulse rate in the predetermined area of the touch panel display 23. Further, the display control unit 107 controls the timing of displaying the systolic blood pressure value, the diastolic blood pressure value, and the pulse rate in the predetermined area of the touch panel display 23. Furthermore, the display control unit 107 instructs the display unit 108 to end the display of the internal pressure of the sensing cuff 15 displayed on the touch panel display 23. Upon receiving the instruction from the display control unit 107, the display unit 108 displays the systolic blood pressure value, the diastolic blood pressure value, and the pulse rate in the predetermined area of the touch panel display 23 instead of displaying the internal pressure of the sensing cuff 15.

Measurement Flow of Cardiac Action Potential in Biological Information Measurement Device 10

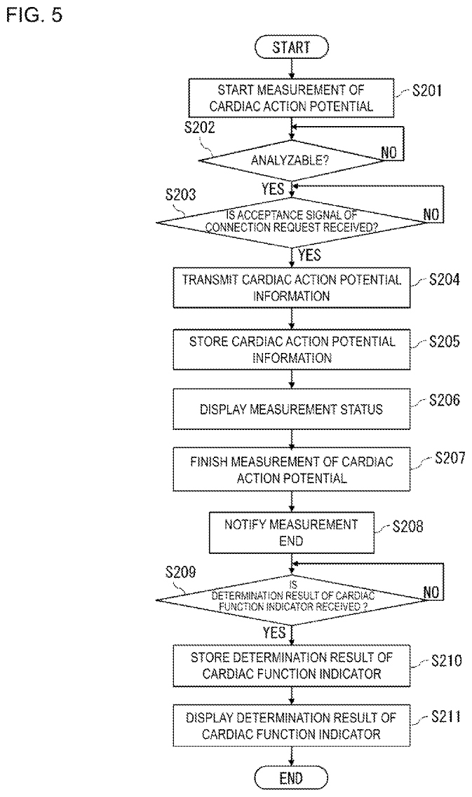

FIG. 5 illustrates an overview of a measurement flow of the cardiac action potential in the biological information measurement device 10.

S201

In step S201, the electrocardiogram control unit 101 is notified from the operation reception unit 106 that the operation input has been performed on the touch panel display 23. Then, the electrocardiogram control unit 101 starts measuring a cardiac action potential. In other words, the electrocardiogram control unit 101 controls a component such as the switch disposed in the electrocardiogram measurement circuit 22 such that the potential difference between the electrode 20A with a potential of the electrode 20B as a reference potential and the electrode 21 can be measured.

S202

In step S202, the electrocardiographic signal quality determination unit 102 acquires, from the electrocardiogram measurement circuit 22, potential difference information between the electrode 20A with a potential of the electrode 20B as a reference potential and the electrode 21. Then, the electrocardiographic signal quality determination unit 102 determines whether the cardiac action potential information can be analyzed. When it is determined that the cardiac action potential information can be analyzed, the electrocardiographic signal quality determination unit 102 inputs the information into the communication unit 105. On the other hand, when it is determined that the cardiac action potential information cannot be analyzed, the electrocardiographic signal quality determination unit 102 acquires again, from the electrocardiogram measurement circuit 22, the potential difference information between the electrode 20A with a potential of the electrode 20B as a reference potential and the electrode 21.

S203

In step S203, the communication unit 105 receives the input of the cardiac action potential information in step S202 and transmits a connection request signal to the external device. Then, the communication unit 105 determines whether an acceptance signal of the connection request is received from the external device.

S204

In step S204, when it is determined that the communication unit 105 has received the acceptance signal of the connection request from the external device, the cardiac action potential information is transmitted to the external device.

On the other hand, when the communication unit 105 has not received the acceptance signal of the connection request from the external device for a predetermined period of time, the communication unit 105 instructs the storage unit 104 to temporarily store the cardiac action potential information in the storage device. Upon receiving the acceptance signal of the connection request from the external device, the communication unit 105 transmits, to the external device, the cardiac action potential information temporarily stored in the storage device.

Note that the storage unit 104 may temporarily store, in the main storage device 26, a part for a predetermined period of time of the potential difference information acquired by the electrocardiographic signal quality determination unit 102 in step S202. Then, in step S204, a part for the predetermined period of time of the potential difference information may be collectively transmitted to the external device. Alternatively, the processing from step S202 to step S204 may be performed by streaming processing.

S205

In step S205, the electrocardiogram control unit 101 instructs the storage unit 104 to store the cardiac action potential information in the storage device. Upon receiving the instruction from the electrocardiogram control unit 101, the storage unit 104 stores the cardiac action potential information in the storage device.

S206

In step S206, the electrocardiogram control unit 101 instructs the display control unit 107 to display a measurement status on the cardiac action potential on the touch panel display 23. Upon receiving the instruction from the electrocardiogram control unit 101, the display control unit 107 instructs the display unit 108 to display, in the predetermined area of the touch panel display 23, the measurement status on the cardiac action potential.

The display unit 108 displays the measurement status on the cardiac action potential on the touch panel display 23 in accordance with the instruction from the display control unit 107.

S207

In step S207, when a predetermined period elapses from the start of the measurement of the cardiac action potential in step S201, the electrocardiogram control unit 101 ends the measurement of the cardiac action potential. In other words, the electrocardiogram control unit 101 controls a component such as the switch disposed in the electrocardiogram measurement circuit 22 such that the potential difference between the electrode 20A with a potential of the electrode 20B as a reference potential and the electrode 21 is not measured.

S208

In step S208, the communication unit 105 notifies the external device that the measurement of the cardiac action potential information is finished. Note that the external device is a destination device to which the cardiac action potential information is transmitted in step S203.

S209

In step S209, the communication unit 105 determines whether the determination result of the cardiac function indicator has been received from the external device. When the communication unit 105 determines that the determination result of the cardiac function indicator has been received from the external device, processing goes to step S210.

S210

In step S210, the storage unit 104 stores, in the storage device, the determination result of the cardiac function indicator received in step S209.

S211

In step S211, the display control unit 107 instructs the display unit 108 to display the determination result of the cardiac function indicator received in step S209 in the predetermined area of the touch panel display 23. Upon receiving the instruction from the display control unit 107, the display unit 108 displays the determination result of the cardiac function indicator in the predetermined area of the touch panel display 23.

Display Flow of Cardiac Action Potential in Smartphone 50

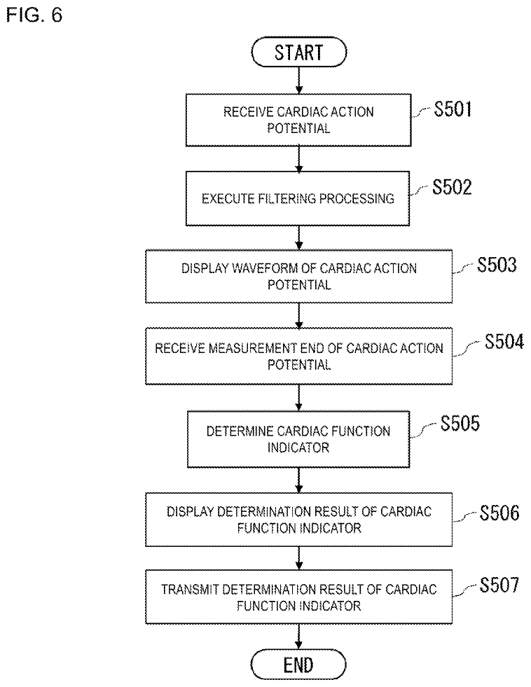

FIG. 6 illustrates an overview of a display flow of the cardiac action potential in the smartphone 50.

S501

In step S501, the communication unit 501 receives the cardiac action potential information transmitted from the external device. Also, such cardiac action potential information is continuously received. Thus, the received cardiac action potential information can form a waveform when being shown graphically.

S502

In step S502, the filter unit 502 executes filtering processing to remove a noise component in the waveform of the cardiac action potential received in step S501. Additionally, the storage unit 505 stores the filtered cardiac action potential information in the storage device.

S503

In step S503, the display unit 504 displays, on the touch panel display 51, the waveform of the cardiac action potential from which noise is removed in step S502.

S504

In step S504, the communication unit 501 receives, from the external device, information indicating that the measurement of the cardiac action potential information is finished.

S505

In step S505, the determination unit 503 determines a cardiac function indicator. In other words, the determination unit 503 acquires, by analysis with a predetermined algorithm, a frequency spectrum from the waveform of the filtered cardiac action potential information stored in the storage device in step S502. Then, whether the cardiac function indicator is abnormal is determined based on the frequency spectrum.

S506

In step S506, the display unit 504 displays a determination result of the cardiac function indicator in step S505 in a predetermined area of the touch panel display 51.

S507

In step S507, the communication unit 501 transmits the determination result of the cardiac function indicator in step S505 to the external device.

Operation Example of Biological Information Measurement System 1

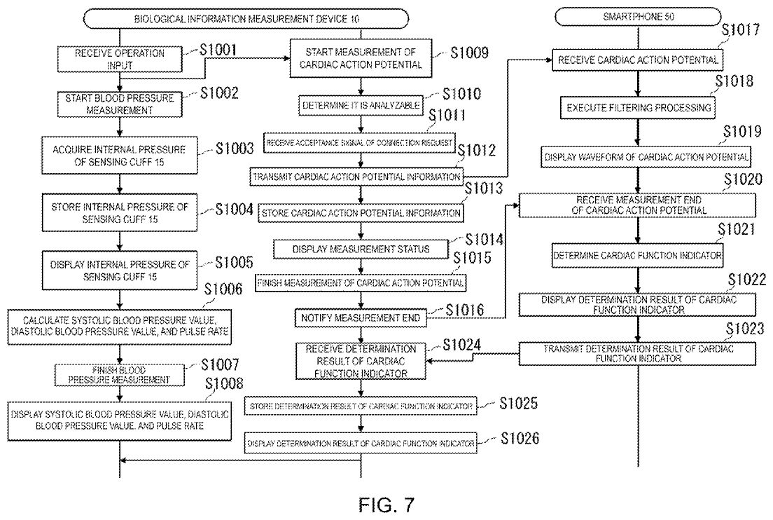

FIG. 7 illustrates an example of a biological information measurement flow executed by the entire biological information measurement system 1.

S1001

In step S1001, the measurement subject fixes the biological information measurement device 10 by wrapping the belt 11 around one arm. Then, the measurement subject taps the touch panel display 23 with a finger. Thereafter, the operation reception unit 106 receives the operation input. Then, the operation reception unit 106 notifies the blood pressure measurement control unit 103 and the electrocardiogram control unit 101 that the operation input has been performed.

S1002

In step S1002, the measurement of blood pressure is started. More specifically, the blood pressure measurement control unit 103 notified of the operation input in step S1001 generates a control signal for reducing the valve opening degree of the exhaust valve 19 to close the exhaust valve 19. Then, the blood pressure measurement control unit 103 generates a drive signal for driving the pump 16 to feed a predetermined amount of air into the compression cuff 12 and the sensing cuff 15 fora predetermined period of time. Further, the blood pressure measurement control unit 103 generates a drive signal for stopping the driving of the pump 16 after the predetermined period of time elapses. Furthermore, the blood pressure measurement control unit 103 generates a control signal for gradually increasing the valve opening degree of the exhaust valve 19 after a predetermined period of time elapses. Thereafter, the blood pressure measurement control unit 103 transmits the generated control signal and drive signal to the exhaust valve 19 and the pump 16.

S1003

In step S1003, when the pump 16 receives the drive signal generated in step S1002, the predetermined amount of air is fed from the pump 16 to the compression cuff 12 and the sensing cuff 15. When the exhaust valve 19 receives the control signal generated in step S1002, the valve opening degree of the exhaust valve 19 is reduced to close the exhaust valve 19. Accordingly, the compression cuff 12 and the sensing cuff 15 continue to inflate. Thus, the sensing cuff 15 is pressed against the arm of the measurement subject. In addition, the degree of pressing by the sensing cuff 15 against the arm of the measurement subject continues to increase. The blood pressure measurement control unit 103 acquires internal pressure of the sensing cuff via the pressure sensor 18 while the sensing cuff 15 continues to press the arm of the measurement subject as just described. After the predetermined period of time elapses, the driving of the pump 16 is stopped, and the valve opening degree of the exhaust valve 19 is gradually increased. Then, the blood pressure measurement control unit 103 continues to acquire the internal pressure of the sensing cuff 15 via the pressure sensor 18 during such a period of time. Note that step S1003 is an example of a “measuring step” of the present disclosure.

S1004

In step S1004, the blood pressure measurement control unit 103 instructs the storage unit 104 to store, in the storage device, the internal pressure of the sensing cuff 15 sequentially acquired via the pressure sensor 18 in step S1003. Upon receiving the instruction, the storage unit 104 stores the internal pressure of the sensing cuff 15 in the storage device.

S1005

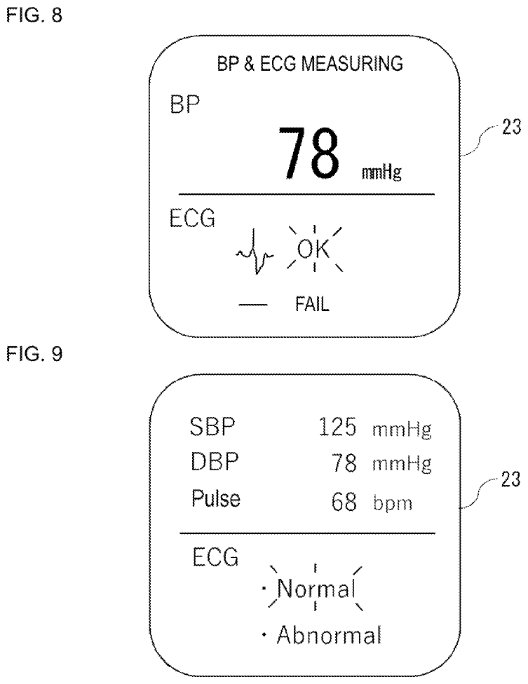

In step S1005, the blood pressure measurement control unit 103 instructs the display control unit 107 to display, on the touch panel display 23, the internal pressure of the sensing cuff 15 sequentially acquired via the pressure sensor 18 in step S1003. The display control unit 107 receives internal pressure information of the sensing cuff 15 from the blood pressure measurement control unit 103 and instructs the display unit 108 to display the internal pressure information in a predetermined area (for example, the upper half) of the touch panel display 23. Then, upon receiving the instruction from the display control unit 107, the display unit 108 displays the internal pressure information of the sensing cuff 15 on the touch panel display 23. FIG. 8 illustrates an overview of the touch panel display 23 in which the internal pressure information of the sensing cuff 15 is displayed in the upper half of the screen.

S1006

In step S1006, the blood pressure measurement control unit 103 calculates a systolic blood pressure value and a diastolic blood pressure value by using the internal pressure of the sensing cuff 15 sequentially acquired via the pressure sensor 18 in step S1003 (an example of a “first processing step” of the present disclosure). In other words, for example, when predetermined vibrations are detected in the internal pressure of the sensing cuff 15 in a process in which the valve opening degree of the exhaust valve 19 is gradually increased, the internal pressure of the sensing cuff 15 at that time is determined as the systolic blood pressure value. Further, when the predetermined vibrations are not detected in the internal pressure of the sensing cuff 15 after the systolic blood pressure value is detected, the internal pressure of the sensing cuff 15 at that time is determined as the diastolic blood pressure value. Furthermore, the blood pressure measurement control unit 103 calculates a pulse rate per minute by using the frequency of the internal pressure of the sensing cuff 15 during the time period from the detection of the systolic blood pressure value to the detection of the diastolic blood pressure value. Then, the blood pressure measurement control unit 103 instructs the storage unit 104 to store the calculated systolic blood pressure value, diastolic blood pressure value, and pulse rate in the storage device. Upon receiving the instruction, the storage unit 104 stores the systolic blood pressure value, the diastolic blood pressure value, and the pulse rate in the storage device.

S1007

In step S1007, when the blood pressure measurement control unit 103 calculates the systolic blood pressure value and the diastolic blood pressure value, the blood pressure measurement control unit 103 determines that the blood pressure measurement is finished. Then, the blood pressure measurement control unit 103 instructs the display control unit 107 to display, on the touch panel display 23, the systolic blood pressure value, the diastolic blood pressure value, and the pulse rate stored in the storage device.

S1008

In step S1008, the display control unit 107 that has received the instruction from the blood pressure measurement control unit 103 in step S1007 instructs the display unit 108 to read out the systolic blood pressure value, the diastolic blood pressure value, and the pulse rate from the storage device and to display the systolic blood pressure value, the diastolic blood pressure value, and the pulse rate in the upper half area of the touch panel display 23. FIG. 9 is an example of a schematic diagram of the touch panel display 23 on which the systolic blood pressure value, the diastolic blood pressure value, and the pulse rate are displayed as just described.

In addition, the display control unit 107 instructs the display unit 108 to start displaying the systolic blood pressure value, the diastolic blood pressure value, and the pulse rate illustrated in FIG. 9. Note that the display control unit 107 instructs the display unit 108 to simultaneously start displaying the determination result of the cardiac function indicator in step S1026 described below. Upon receiving the instruction from the display control unit 107, the display unit 108 displays the systolic blood pressure value, the diastolic blood pressure value, and the pulse rate in the upper half area of the touch panel display 23 as illustrated in FIG. 9, and displays the determination result of the cardiac function indicator (described below) in the lower half area. Note that step S1008 is an example of a “displaying step” of the present disclosure.

S1009

In step S1009, the electrocardiogram control unit 101 notified from the operation reception unit 106 of the operation input of the touch panel display 23 in step S1001 starts measuring a cardiac action potential. In other words, the electrocardiogram control unit 101 controls a component such as the switch disposed in the electrocardiogram measurement circuit 22 such that the potential difference between the electrode 20A with a potential of the electrode 20B as a reference potential and the electrode 21 can be measured. In addition, the measurement subject touches the electrode 20A and the electrode 20B with fingers of the arm on the opposite side to the arm on which the biological information measurement device 10 is attached.

S1010

In step S1010, the electrocardiographic signal quality determination unit 102 acquires, from the electrocardiogram measurement circuit 22, a potential difference between the electrode 20A with a potential of the electrode 20B as a reference potential and the electrode 21. Then, the electrocardiographic signal quality determination unit 102 determines that the cardiac action potential information can be analyzed with respect to the potential difference. Then, the electrocardiographic signal quality determination unit 102 determines the potential difference as a cardiac action potential. Thereafter, the electrocardiographic signal quality determination unit 102 inputs into the communication unit 105 the determination that the cardiac action potential information can be analyzed. Note that step S1010 is an example of the “measuring step” of the present disclosure.

S1011

In step S1011, the communication unit 105 receives the input of the cardiac action potential information in step S1010 and transmits a connection request to the communication unit 501 of the smartphone 50. Then, the communication unit 105 receives, from the communication unit 501 of the smartphone 50, an acceptance signal of the connection request.

S1012

In step S1012, the communication unit 105 that has received the connection request signal from the communication unit 501 in step S1011 transmits the cardiac action potential information to the communication unit 501. Such cardiac action potential information is continuously acquired via the electrocardiogram measurement circuit 22 and is transmitted to the communication unit 501. Note that step S1011 is an example of a “first transmitting step” of the present disclosure.

S1013

In step S1013, the electrocardiogram control unit 101 instructs the storage unit 104 to store the cardiac action potential information in the storage device. Upon receiving the instruction from the electrocardiogram control unit 101, the storage unit 104 stores the cardiac action potential information in the storage device.

S1014

In step S1014, the electrocardiogram control unit 101 instructs the display control unit 107 to display a measurement status on the cardiac action potential on the touch panel display 23. Upon receiving the instruction from the electrocardiogram control unit 101, the display control unit 107 instructs the display unit 108 to display the measurement status on the cardiac action potential in the lower half of the touch panel display 23 as illustrated in FIG. 8.

More specifically, the display control unit 107 instructs the display unit 108 to display the characters “OK” and “FAIL” on the lower half of the touch panel display 23. Further, the display control unit 107 instructs the display unit 108 to display waveforms corresponding to “OK” and “FAIL”. Furthermore, when the measurement status on the cardiac action potential is normal, the display control unit 107 instructs the display unit 108 to blink the “OK” portion. On the other hand, when the measurement status on the cardiac action potential is abnormal, the display control unit 107 instructs the display unit 108 to blink the “FAIL” portion.

The display unit 108 displays the aforementioned information on the touch panel display 23 in accordance with the instruction from the display control unit 107. FIG. 8 illustrates a case where the measurement status on the cardiac action potential is normal. Note that as illustrated in FIG. 8, the internal pressure of the sensing cuff 15 is displayed in the upper half of the touch panel display 23 as a result of the execution of processing in step S1005.

S1015

In step S1015, when a predetermined period of time elapses from the start of the measurement of the cardiac action potential in step S1009, the electrocardiogram control unit 101 ends the measurement of the cardiac action potential. In other words, the electrocardiogram control unit 101 controls a component such as the switch disposed in the electrocardiogram measurement circuit 22 such that the potential difference between the electrode 20A with a potential of the electrode 20B as a reference potential and the electrode 21 is not measured.

S1016

In step S1016, the communication unit 105 transmits, to the communication unit 501 of the smartphone 50, information indicating that the measurement of the cardiac action potential information is finished.

S1017

In step S1017, the communication unit 501 of the smartphone 50 receives the cardiac action potential information transmitted from the communication unit 105 of the biological information measurement device 10 in step S1012. Also, such cardiac action potential information is continuously received. Thus, the received cardiac action potential information can form a waveform when being shown graphically. Note that step S1017 is an example of a “first receiving step” of the present disclosure.

S1018

In step S1018, the filter unit 502 executes filtering processing to remove a noise component from the waveform of the cardiac action potential received in step S1017. Additionally, the storage unit 505 stores the filtered cardiac action potential information in the storage device. Note that step S1018 is an example of a “second processing step” of the present disclosure.

S1019

In step S1019, the display unit 504 displays, on the touch panel display 51, the waveform of the cardiac action potential from which noise is removed in step S1018.

S1020

In step S1020, the communication unit 501 receives a notice of the completion of the measurement of the cardiac action potential information transmitted from the communication unit 105 of the biological information measurement device 10 in step S1016.

S1021

In step S1021, the determination unit 503 determines a cardiac function indicator. In other words, the determination unit 503 acquires, for example, heart rate information from the waveform of the filtered cardiac action potential information stored in the storage device in step S1018. Then, the determination unit 503 determines whether the heart rate information is higher than a predetermined value. In addition, the determination unit 503 determines, for example, by performing FFT analysis on the waveform of the cardiac action potential information whether the cardiac rhythm is abnormal. Note that step S1021 is an example of the “second processing step” of the present disclosure.

S1022

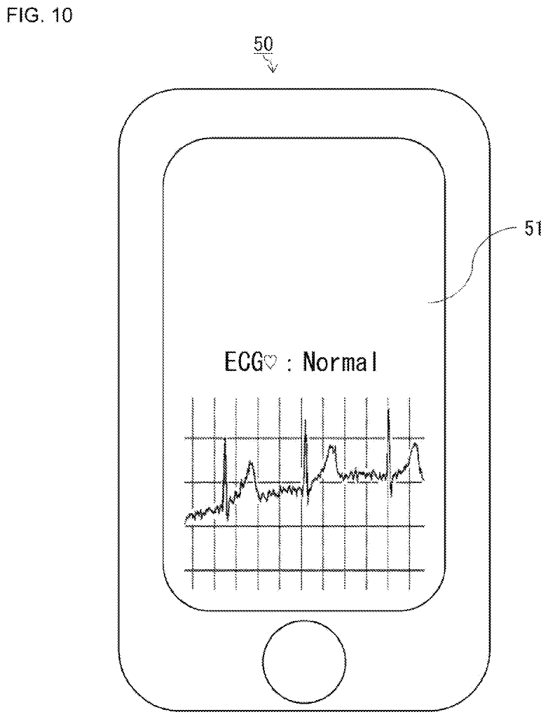

In step S1022, the display unit 504 displays the determination result of the cardiac function indicator in step S1021 on the touch panel display 51. FIG. 10 illustrates an overview of the determination result of the cardiac function indicator displayed on the touch panel display 51. Here, as illustrated in FIG. 10, the display unit 504 displays the determination result of the cardiac function indicator in an area different from the area in which the waveform of the cardiac action potential is displayed. Note that in the example illustrated in FIG. 10, a case where the determination result of the cardiac function indicator is normal (Normal) is illustrated.

S1023

In step S1023, the communication unit 501 transmits the determination result of the cardiac function indicator in step S1021 to the communication unit 105 of the biological information measurement device 10. Note that step S1023 is an example of a “second transmitting step” of the present disclosure.

S1024

In step S1024, the communication unit 105 of the biological information measurement device 10 receives the determination result of the cardiac function indicator transmitted from the communication unit 501 of the smartphone 50 in step S1023. Note that step S1024 is an example of a “second receiving step” of the present disclosure.

S1025

In step S1025, the storage unit 104 stores, in the storage device, the determination result of the cardiac function indicator received in step S1024.

S1026

In step S1026, the display control unit 107 instructs the display unit 108 to display, in the lower half of the touch panel display 23, the determination result of the cardiac function indicator received in step S1024. As described above, the display control unit 107 instructs the display unit 108 to start displaying the determination result of the cardiac function indicator at the same time as displaying the systolic blood pressure value, the diastolic blood pressure value, and the pulse rate (step S1008).

More specifically, as illustrated in FIG. 9, the display control unit 107 instructs the display unit 108 to display the characters “Normal” and “Abnormal” in the lower half of the touch panel display 23.

Further, for example, when the determination result of the cardiac function indicator is normal, the display control unit 107 instructs the display unit 108 to blink the “Normal” portion. Furthermore, for example, when the determination result of the cardiac function indicator is abnormal, the display control unit 107 instructs the display unit 108 to blink the “Abnormal” portion. The display unit 108 displays the aforementioned information on the touch panel display 23 in accordance with the instruction from the display control unit 107. FIG. 9 illustrates a case where the determination result of the cardiac function indicator is normal. Note that step S1026 is an example of the “displaying step” of the present disclosure.

### Actions and Effects

According to the biological information measurement system 1 described above, computational costs of the biological information measurement device 10 are saved as compared with a case where the filtering processing on the waveform of the cardiac action potential is executed in the biological information measurement device 10. Accordingly, the MCU 24 in which calculation performance is suppressed as compared with the processor of the smartphone 50 can be adopted in a processor mounted on the biological information measurement device 10. Therefore, an increase in size of the processor of the biological information measurement device 10 and an increase in power consumption can be suppressed. Consequently, as illustrated in FIG. 1 and FIG. 2, an increase in size of the biological information measurement device 10 on which components that can measure two types of biological information, i.e., blood pressure and a cardiac action potential are mounted is suppressed. As a result, the biological information measurement device 10 can be easily carried, and convenience is improved.

Further, according to the biological information measurement system 1 as described above, the filtering processing on the waveform of the cardiac action potential and the determination processing of the cardiac function indicator, which relatively require computational costs are executed in the smartphone 50. Accordingly, the processing speed of the entire system is improved compared with a case where these types of processing are executed in the biological information measurement device 10. Therefore, the biological information measurement device 10 can display the systolic blood pressure value, the diastolic blood pressure value, and the pulse rate (step S1008), and the determination result of the cardiac function indicator (step S1026) in real time and simultaneously (see FIG. 9). Consequently, the measurement subject can simultaneously confirm two different pieces of information of the blood pressure value and the cardiac function indicator. As a result, the convenience of the biological information measurement device 10 is improved.

Furthermore, according to the biological information measurement system 1 as described above, even when not carrying the biological information measurement device 10, the measurement subject can check the waveform of the cardiac action potential and the cardiac function indicator displayed on the touch panel display 51 of the smartphone 50.

In addition, according to the biological information measurement system 1 as described above, before the biological information measurement device 10 transmits the cardiac action potential information to the smartphone 50 (step S1012), the biological information measurement device 10 determines, by a simple determination that can be processed even by an inexpensive MCU 24 with low functionality, whether the cardiac action potential information can be analyzed (step S1010). Therefore, according to the biological information measurement system 1 as described above, the occurrence of processing other than the filtering processing (step S1018) and the determination processing of the cardiac function indicator (step S1021) is suppressed in the smartphone 50. Consequently, calculation resources in the smartphone 50 can be concentrated on the filtering processing (step S1018) and the determination processing of the cardiac function indicator (step S1021). As a result, a decrease in processing speed is suppressed.

Moreover, according to the biological information measurement system 1 described above, the internal pressure of the sensing cuff 15 is displayed on the touch panel display 23 of the biological information measurement device 10 in step S1005 as illustrated in FIG. 8. In addition, as illustrated in FIG. 8, the measurement status on the cardiac action potential is displayed in step S1014. Therefore, during the measurement, the measurement subject can recognize whether the blood pressure measurement or the measurement of the cardiac action potential is normally executed. As a result, the measurement subject can perform measurement over again before the measurement result of the blood pressure and the determination result of the cardiac function indicator are displayed on the touch panel display 23 (step S1008 and step S1026).

### § 2 Modified Examples

According to the biological information measurement system 1 described above, the cardiac action potential-related information is transmitted from the biological information measurement device 10 to the smartphone 50, and the filtering processing and the determination processing of the cardiac function indicator are executed in the smartphone 50. However, in the biological information measurement device 10, the filtering processing and the determination processing of the cardiac function indicator may be performed on the cardiac action potential information. Then, the blood pressure information may be transmitted from the biological information measurement device 10 to the smartphone 50, and predetermined processing may be executed on the blood pressure information.

In addition to or instead of the blood pressure and the cardiac action potential, the biological information measurement device 10 may measure pulse waves, percutaneous arterial blood oxygen saturation, action potentials generated from muscle fibers, acceleration of the biological information measurement device 10 in a predetermined direction, transmission or reflection intensity of the light emitted from the biological information measurement device 10 toward the measurement subject, ambient temperature of the biological information measurement device 10, or body temperature of the measurement subject to which the biological information measurement device 10 is attached other than the measurement of the blood pressure and cardiac action potential information described above as an example. Further, in the biological information measurement device 10, at least two or more pieces of information of these pieces of information related to the living body may be measured. Furthermore, predetermined processing may be executed on predetermined information of these pieces of information in the smartphone 50. In addition, a plurality of pieces of measurement information may be transmitted from the biological information measurement device 10 to the smartphone 50 to be subjected to the predetermined processing. The same or similar effects as those of the embodiment can be achieved even by such a biological information measurement system.

Moreover, in the embodiment described above, the filtering processing and the determination processing of the cardiac function indicator are illustrated as processing to be executed on the cardiac action potential information in the smartphone 50; however, the processing in the smartphone 50 is not limited to such an example.

Further, in the embodiment described above, the systolic blood pressure value, the diastolic blood pressure value, the pulse rate, and the determination result of the cardiac function indicator are simultaneously displayed on the touch panel display 23 as illustrated in step S1008 and step S1026; however, these measurement results may not be simultaneously displayed.

Furthermore, the waveform of the cardiac action potential or the determination result of the cardiac function indicator may not be displayed on touch panel display 51 of the smartphone 50.

In addition, in the biological information measurement device 10, the blood pressure measurement flow (step S1002 to step S1007) and a part of the measurement flow of the cardiac action potential (step S1009 to step S1016) may be processed sequentially or may be processed in parallel.

Moreover, an example where two different pieces of biological information of the blood pressure and the cardiac function indicator are displayed on the touch panel display 23 is illustrated in the embodiment described above. However, the output related to the internal pressure of the sensing cuff 15 is acquired (an example of the “first information” and the “second information” of the present disclosure), and the internal pressure of the sensing cuff 15 (an example of a “first information after execution of the first processing related to display” of the present disclosure) and the blood pressure information calculated based on the output related to the internal pressure of the sensing cuff 15 (an example of a “second information after execution of the second processing related to display” of the present disclosure) may be displayed on the touch panel display 23. In such a case, the calculation of blood pressure based on the output related to the internal pressure of the sensing cuff 15 may be executed in the smartphone 50.

In addition, as a replacement for the smartphone 50, a terminal equipped with an MCU having calculation performance equal to or lower than that of the MCU 24 included in the biological information measurement device 10 may be adopted.

Additionally, the structure of the biological information measurement device 10 for measuring blood pressure is not limited to the examples illustrated in FIG. 1 and FIG. 2.

The embodiments and modified examples disclosed above can be combined with each other.

### REFERENCE NUMERALS LIST

1: Biological information measurement system
10: Biological information measurement device
11: Belt
12: Compression cuff
14: Back plate
15: Sensing cuff
16: Pump
17: Drive circuit
18: Pressure sensor
19: Exhaust valve
20A: Electrode
20B: Electrode
21: Electrode
22: Electrocardiogram measurement circuit
23: Touch panel display
25: Communication module
26: Main storage device
27: Auxiliary storage device
28: Power source
29: Arithmetic circuit
50: Smartphone
51: Touch panel display
101: Electrocardiogram control unit
102: Electrocardiographic signal quality determination unit
103: Blood pressure measurement control unit
104: Storage unit
105: Communication unit
106: Operation reception unit
107: Display control unit
108: Display unit
501: Communication unit
502: Filter unit
503: Determination unit
504: Display unit
505: Storage unit

1. A biological information measurement system comprising:
a biological information measurement device; and
a terminal that is portable,
wherein the biological information measurement device comprises:
a measurement unit configured to measure at least first information related to a living body and second information related to the living body;
a first processing unit configured to execute first processing related to display and/or diagnosis on the first information measured by the measurement unit;
a quality determination unit configured to determine whether the second information is analyzable; and
a communication unit configured to communicate the second information determined to be analyzable with the terminal;
the terminal comprises:
a reception unit configured to receive the second information from the biological information measurement device;
a second processing unit configured to execute, in parallel with the first processing, second processing related to display and/or diagnosis on the second information received by the reception unit; and
a transmission unit configured to transmit the second information after the second processing is executed by the second processing unit to the biological information measurement device, and
the biological information measurement device further comprises a first display unit configured to display the first information after the first processing is executed by the first processing unit and the second information transmitted to the biological information measurement device by the transmission unit after the second processing is executed by the second processing unit.

2. The biological information measurement system according to claim 1, wherein the terminal further comprises a second display unit configured to display the second information on which the second processing is executed by the second processing unit.

3. A biological information measurement recording medium configured to cause a computer to execute:
a measuring step of measuring at least first information related to a living body and second information related to the living body in a biological information measurement device;
a first processing step of executing first processing related to display and/or diagnosis on the first information measured in the measuring step in the biological information measurement device;
a quality determining step of determining in the biological information measurement device whether the second information measured in the measuring step is analyzable;
a first transmitting step of transmitting the second information determined to be analyzable to a terminal that is portable in the biological information measurement device;
a first receiving step of receiving, in the terminal, the second information transmitted from the biological information measurement device in the first transmitting step;
a second processing step of executing in parallel with the first processing, in the terminal, second processing related to display and/or diagnosis on the second information received in the first receiving step;
a second transmitting step of transmitting, in the terminal, the second information on which the second processing is executed in the second processing step to the biological information measurement device;
a second receiving step of receiving, in the biological information measurement device, the second information on which the second processing is executed, the second information transmitted from the terminal in the second transmitting step; and
a displaying step of displaying, in the biological information measurement device, the first information after the executing the first processing and the second information after the executing the second processing received in the second receiving step.

4. A biological information measurement device comprising:
a measurement unit configured to measure at least first information related to a living body and second information related to the living body;
a first processing unit configured to execute first processing related to display and/or diagnosis on the first information measured by the measurement unit;
a quality determination unit configured to determine whether the second information measured by the measurement unit is analyzable;
a communication unit configured to communicate the second information determined to be analyzable with an external device including a second processing unit configured to execute, in parallel with the first processing, second processing related to display and/or diagnosis on the second information; and
a display unit configured to display the first information on which the first processing is executed by the first processing unit and the second information after execution of the second processing by the external device.

5. The biological information measurement device according to claim 4, wherein the first information includes blood pressure information, and
the second information includes cardiac action potential information.

6. The biological information measurement device according to claim 5, wherein the second processing includes filtering processing on a waveform of the cardiac action potential and determination processing related to a cardiac function.

7. The biological information measurement device according to claim 4, wherein the second information includes at least one piece of information about blood pressure, a pulse wave, percutaneous arterial blood oxygen saturation, an action potential generated from a muscle fiber, acceleration of the biological information measurement device in a predetermined direction, transmission or reflection intensity of light emitted from the biological information measurement device, ambient temperature of the biological information measurement device, or body temperature of a measurement subject on which the biological information measurement device is attached.

8. The biological information measurement device according to claim 4, wherein the display unit synchronizes timing of displaying the first information on which the first processing is executed by the first processing unit and timing of displaying the second information on which the second processing is executed by the second processing unit.

9. The biological information measurement device according to claim 5, wherein the second information includes at least one piece of information about blood pressure, a pulse wave, percutaneous arterial blood oxygen saturation, an action potential generated from a muscle fiber, acceleration of the biological information measurement device in a predetermined direction, transmission or reflection intensity of light emitted from the biological information measurement device, ambient temperature of the biological information measurement device, or body temperature of a measurement subject on which the biological information measurement device is attached.

10. The biological information measurement device according to claim 6, wherein the second information includes at least one piece of information about blood pressure, a pulse wave, percutaneous arterial blood oxygen saturation, an action potential generated from a muscle fiber, acceleration of the biological information measurement device in a predetermined direction, transmission or reflection intensity of light emitted from the biological information measurement device, ambient temperature of the biological information measurement device, or body temperature of a measurement subject on which the biological information measurement device is attached.

11. The biological information measurement device according to claim 5, wherein the display unit synchronizes timing of displaying the first information on which the first processing is executed by the first processing unit and timing of displaying the second information on which the second processing is executed by the second processing unit.

12. The biological information measurement device according to claim 6, wherein the display unit synchronizes timing of displaying the first information on which the first processing is executed by the first processing unit and timing of displaying the second information on which the second processing is executed by the second processing unit.

13. The biological information measurement device according to claim 7, wherein the display unit synchronizes timing of displaying the first information on which the first processing is executed by the first processing unit and timing of displaying the second information on which the second processing is executed by the second processing unit.
